(19) United States
(12) Patent Application Publication
(10) Pub. No.: US 2020/0396958 A1
(43) Pub. Date: Dec. 24, 2020

(54) TEMPERATURE CONTROLLED BED ASSEMBLY FOR PETS

(71) Applicant: George B Eways, Corona, CA (US)

(72) Inventor: George B Eways, Corona, CA (US)

(21) Appl. No.: 17/011,649

(22) Filed: Sep. 3, 2020

(51) Int. Cl. A01K 1/015; A01K 1/035; A01K 29/00; A61L 9/12

(52) U.S. Cl. CPC A01K 1/0158; A01K 1/0353; A61L 2209/11; A61L 9/12; A01K 29/005

(57) ABSTRACT

The present invention is directed to a bed assembly for protecting and comforting pets against extreme weather conditions. The bed assembly includes a frame having a top side and a wall, the top side having a plurality of apertures. A temperature control unit is enclosed in the frame, the temperature control comprises a heating unit, a cooling unit, and at least one fan, the at least one fan positioned to convey conditioned air through the plurality of apertures, the conditioned air is air heated by the heating unit or cooled by the cooling unit. A control unit can be operably coupled to the temperature control unit; and a pressure sensor can be mounted on the frame and configured to detect the presence of a pet on the frame, the pressure sensor coupled to the control unit. Wherein the control unit is configured to receive a signal from the pressure sensor, and upon receiving the signal, activate the temperature control unit to provide conditioned air.

### FIELD OF INVENTION

The present invention generally relates to a bed assembly for a pet, and in particular, the present invention relates to a temperature-controlled bed assembly for pets.

### BACKGROUND

Animals are susceptible to extreme temperatures. In particular, the extreme changes in temperature may prove unhealthy for pets. In hot weather, a pet may seek out a cool spot in the house. For example, it is common to see a dog sleeping in a bathtub in hot weather. Similarly, in chilly weather, the pet may seek a warm spot.

The bed and bedding, generally available for pets are focused on softness or appearance of the bed, for example, sofa beds are commercially available that are aesthetic in appearance and soft for the pets.

However, there is an urgent need for a temperature-controlled bed for pets that could provide both protection and comfort against extreme weather.

### SUMMARY OF THE INVENTION

The principal object of the present invention is therefore directed to temperature-controlled bed assembly for pets.

It is a further object of the present invention that the bed assembly can provide protection against both the cold and hot weathers,

It is an additional object of the present invention that the user can preset the temperature of conditioned air.

It is another object of the present invention that the bed assembly can intelligently control the temperature.

It is still another object of the present invention that the user can set the temperature remotely.

It is a further object of the present invention that the bed assembly can also provide changeable essences for the pet and to hide any pet odor.

In one aspect, the present invention is directed to a bed assembly for a pet. The definition of a pet is well known in the art and includes dogs and cats. Perhaps, dogs and cats are the two most popular pets. The bed assembly disclosed herein includes a frame, the frame having a length, a width, and a height. The length and the width define an area of the bed for sitting or sleeping. The bed assembly comprises a heating unit and a cooling unit both enclosed in the frame. The bed assembly further comprises a control unit operably coupled to the cooling unit and the heating unit. The control assembly including a networking circuitry for connecting to a network.

The bed assembly further includes a control panel coupled to the outer surface of the frame that includes the controls for setting the temperature and activating the unit. A thermometer, for example, an infrared-based thermometer can also be provided outside the frame to detect the environment temperature. The thermometer can also connect to the control unit for controlling the operation of the heating and cooling unit. The bed assembly may also comprise a camera coupled to the control unit and positioned to capture images of the pet lying on the bed assembly.

In one aspect, the bed assembly further includes a pressure/weight sensor configured for detecting the presence of a pet on the bed assembly. The pressure sensor coupled to the control unit, wherein the control unit can operate the temperature regulating units including the heating unit and the cooling unit on receiving a signal from the pressure sensor.

### BRIEF DESCRIPTION OF THE DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a block diagram showing the components of the bed assembly, according to an exemplary embodiment of the present invention.

FIG. 2 is an environment diagram of the bed assembly showing the control unit connected to a user device through a network

FIG. 3 shows a perspective view of the bed assembly, according to an exemplary embodiment of the present invention.

### DETAILED DESCRIPTION

Subject matter will now be described more fully hereinafter. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as devices and methods of use thereof. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is for the purpose of describing particular embodiments only and is not intended to be limiting of embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely for the purpose of illustrating the general principles of the invention, since the scope of the invention will be best defined by the allowed claims of any resulting patent.

The following detailed description is described with reference to the drawings, wherein like reference numerals are used to refer to like elements throughout. In the following description, for purposes of explanation, specific details may be set forth in order to provide a thorough understanding of the subject innovation. It may be evident, however, that the claimed subject matter may be practiced without these specific details.

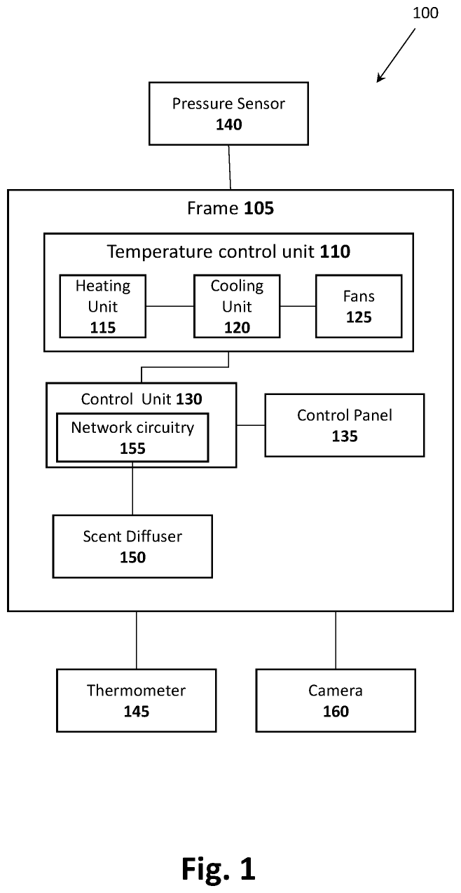

The present invention is directed to a bed assembly for pets, in particular to a temperature-controlled bed assembly that can protect a pet from extreme temperature conditions including the hot and wintry weather. The pet can be any animal known to a skilled person as a pet and particularly includes the dogs and cats. The bed assembly provides a comfortable surface to a pet for relaxing and sleeping. Referring to FIG. 1, the bed assembly 100 includes a frame 105 which forms the bed. The frame 105 is having a length, width, and height. The length and width define an area of the bed available to the pet for lying down. The bed is typically cuboidal in shape, wherein the length and width can be the same or different. The edges can be rounded. It is however obvious that the frame can be provided in different shapes, including rhombus, round, and like. All such shapes of the frame are within the scope of the present invention. The frame can be made of wood, metal, plastic, and like material known to a skilled person for manufacturing furniture items. Moreover, the frame can be manufactured in assorted sizes to suit animals of assorted sizes. Referring again to FIG. 1, which shows the bed assembly 100 including a temperature control unit 110. The temperature control unit can include a heating unit 115 and a cooling unit 120. Either the heating unit or the cooling unit can be activated to provide conditioned air to the bed surface. For example, in hot weather, the cooling unit can be activated to provide cool air to the bed surface, while in the chilly weather, the heating unit can be activated to provide warm air to the bed surface. The bed assembly may also include fans 125 for conveying the hot or cool air through the apertures in the top side of the frame. The temperature control unit can be coupled to a control unit 130, which controls the operation of the heating unit and the cooling unit. The control unit 130 can also be encased in the frame.

The bed assembly may further include a control panel 135 that can for setting the desired temperature. For example, the control panel may have a small display showing the set temperature. A pair of buttons for setting the desired temperature, wherein one button increases the temperature while the other decreases. The control panel may also allow the user to activate either of the heating unit or the cooling unit. The control panel can mount on the outer side of the frame, such as it is accessible to a user. Alternatively, the control panel can connect to the control unit through an extended wire. The control panel can also be wirelessly connected to the control unit, for example, through Bluetooth.

The bed assembly may further include a pressure sensor 140. The pressure sensor 140, also referred herein as the weight sensor can detect the presence of the pet on the bed, the bed formed by the frame. The pressure sensor 140 can be mounted on top of the frame, maybe below the beddings. The top surface of the frame can include covers and cushions for the pet. Such cover and cushions are known in the art for bedding of pets. Moreover, the top side of the frame is having multiple apertures for receiving the conditioned air from the temperature control unit 110. These apertures can be continued with the cushioning on the bed. The pressure sensor can be configured below the cushioning and over the top surface of the frame. The pressure sensor can be calibrated according to the weight of the pet, thus avoiding any false signals.

The bed assembly may also include a temperature sensor, for example, a room thermometer can be used. The temperature sensor can be used for taking the temperature of the room. Such sensors are known in the art for sensing the temperature of a room. The temperature sensor can couple with the control unit, wherein the control unit can receive the temperature readings from the temperature sensor. The control unit upon receiving the temperatures can automatically control the temperature control unit to activate either the heating unit or the cooling unit. For example, the control unit upon detecting that the room temperature is too high can activate the cooling unit which can then provide cooler air to the bed. Alternatively, the temperature sensor can also be embedded in the bedding, thus detecting the temperature of the bedding. Based on the temperature of the bedding, the control unit can activate the temperature control unit to bring the temperature of the bedding within the predetermined range of the temperature.

The bedding assembly may also include a scent diffuser 150, which may also enclose in the frame. The scent diffuser may provide essence that can be both pleasant and mask the odor of the bedding. The scent diffuser may include distinct essences, wherein one or more essences can be interchangeably used. Alternatively, the user can select the desired essence. Else, the control unit can also decide the essence to be used. The control unit may also include a network circuitry 155 for connecting to an external network.

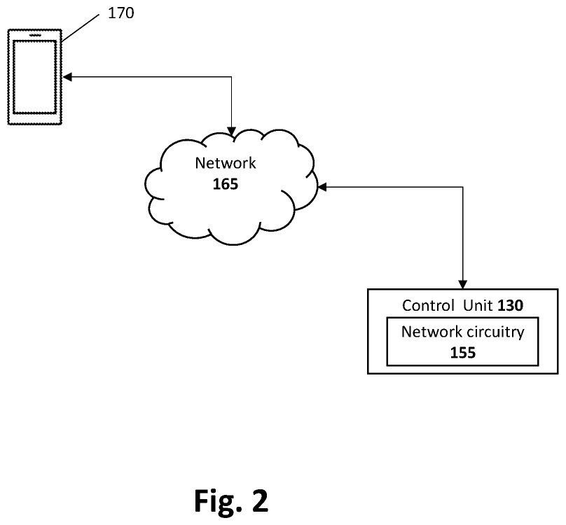

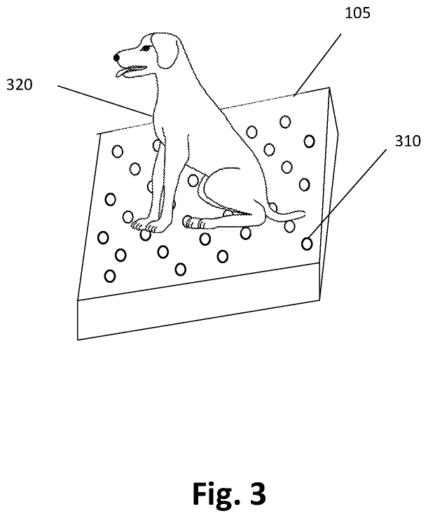

Referring to FIG. 2, the control unit 130 including the network circuitry 155 can also connect to an external network 165. The external network can be a local network, wide area network, internet, Wi-Fi, and like. Such networks for connecting electronic devices are known in the art, and such networks are within the scope of the present invention. A user device 170 can remotely connect with the control unit 130 for both checking the status of the bed assembly and also to command the control unit. Through the user device, a user can override the control unit for setting the desired temperature range for the bedding, activate the temperature control unit, activate the scent diffuser, or select the specific essence. The user can also see the temperature of the bedding as detected by the temperature sensor. The user can see the bedding area through a camera. The camera can also couple with the control unit. FIG. 2 shows a camera 160 couple to the control unit 130. The control unit can also include appropriate image recognition algorithms and artificial intelligence algorithms that can analyze the feed received from the camera. The control unit can detect the comfort level of the pet lying on the bedding. Based on the detected comfort level of the pet, the control unit can automatically regulate the temperature control unit to bring the comfort level of the pet within the predetermined range of comfort levels. For example, certain body movements of the pet can be analyzed to determine the comfort level of the pet. Or the reaction of the pet to the extreme temperatures can also be used for learning by the control unit. Moreover, the control unit can also learn from the reactions of the pet to different scents and select the scent that makes the pet more comfortable. FIG. 3 shows a perspective view of the frame 105 having multiple apertures 310 and a dog 320 sitting on the frame.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. A bed assembly comprising:
a frame, the frame having a top side and a wall, the top side having a plurality of apertures;
a temperature control unit enclosed in the frame, the temperature control comprises a heating unit, a cooling unit, and at least one fan, the at least one fan positioned to convey conditioned air through the plurality of apertures, the conditioned air is air heated by the heating unit or cooled by the cooling unit;
a control unit operably coupled to the temperature control unit; and
a pressure sensor mounted on the frame and configured to detect the presence of a pet on the frame, the pressure sensor coupled to the control unit, wherein the control unit is configured to:
receive a signal from the pressure sensor,
upon receiving the signal, activate the temperature control unit.

2. The bed assembly of claim 1, further comprising a temperature sensor configured to take temperature of a bedding coupled to the frame, the temperature sensor coupled to the control unit, wherein the control unit is configured to:
receive the temperature from the temperature sensor, and
activate either the heating unit or the cooling unit to bring the temperature of the bedding within a predetermined range.

3. The bed assembly of claim 2, wherein the temperature sensor is an infrared sensor configured in the bedding.

4. The bed assembly of claim 2, wherein a user presets the predetermined range of temperature.

5. The bed assembly of claim 1, wherein the bed assembly further comprises a scent diffuser, the scent diffuser comprises a plurality of scents, the scent diffuser coupled to the control unit, the control unit configured to:
receive a selection for the scent;
activate the scent diffuser to release the selected scent.

6. The bed assembly of claim 1, wherein the control unit further comprises a network circuitry configured for connecting to a network, the control unit configured to connect with a user device through the network, the control unit further configured to receive a temperature from the user device.

7. The bed assembly of claim 1, wherein the bed assembly further comprises a camera coupled to the control unit.

8. A method of regulating temperature of a bedding of a pet, the method comprises:
providing a bedding, the bedding comprises:
a frame, the frame having a top side and a wall, the top side having a plurality of apertures, the bedding mounted on top side of the frame,
a temperature control unit enclosed in the frame, the temperature control comprises a heating unit, a cooling unit, and at least one fan, the at least one fan positioned to convey conditioned air through the plurality of apertures, the conditioned air is air heated by the heating unit or cooled by the cooling unit,
a control unit operably coupled to the temperature control unit, and
a pressure sensor mounted on the frame and configured to detect the presence of a pet on the frame, the pressure sensor coupled to the control unit;
receiving a signal from the pressure sensor indicating the presence of a pet;
upon receiving the signal, activating the temperature control unit for providing the conditioned air.
